SEQUENCE LISTING (1 sequence)

SEQ ID NO: 1 - protein, 178 aa, Homo sapiens
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

(19) United States
(12) Patent Application Publication
(10) Pub. No.: US 2013/0178543 A1
(43) Pub. Date: Jul. 11, 2013

(54) BIOMARKER FOR DIAGNOSING CANCER AND METHOD OF ISOLATING CANCER CELL USING THE SAME

(71) Applicants: Yeon-jeong KIM, Yongin-si (KR); You-sun KIM, Suwon-si (KR); Jeong-gun LEE, Seoul (KR); Sang-hyun BAEK, Hwaseong-si (KR); Jin-mi OH, Suwon-si (KR); Hyo-young JEONG, Incheon (KR)

(72) Inventors: Yeon-jeong KIM, Yongin-si (KR); You-sun KIM, Suwon-si (KR); Jeong-gun LEE, Seoul (KR); Sang-hyun BAEK, Hwaseong-si (KR); Jin-mi OH, Suwon-si (KR); Hyo-young JEONG, Incheon (KR)

(73) Assignees: AJOU UNIVERSITY INDUSTRY COOPERATION FOUNDATION, Suwon-si (KR); SAMSUNG ELECTRONICS CO., LTD., Suwon-si (KR)

(21) Appl. No.: 13/594,610

(22) Filed: Aug. 24, 2012

(30) Foreign Application Priority Data
Jan. 10, 2012 (KR) 10-2012-0003081

(51) Int. Cl. G01N 33/574; C12N 5/095; C40B 30/04; C07K 17/00; A61P 35/00; A61P 35/04; C12N 5/09; G01N 33/577

(52) U.S. Cl. 514/789; 435/325; 435/7.23; 530/391.1; 506/9

(57) ABSTRACT

A method of detecting a cancer stem cell or circulating tumor cell that has undergone epithelial-mesenchymal transition, comprising determining the level of caveolin-1 expressed by a sample cell, and comparing the level of caveolin-1 expressed by the sample cell to a control, wherein higher expression of caveolin-1 by the sample cell indicates that the sample cell is a cancer cell, as well as a method of detecting cancer or metastasis in a subject, and related methods and compositions.

### CROSS-REFERENCE TO RELATED APPLICATIONS

This application claims the benefit of Korean Patent Application No. 10-2012-0003081, filed on Jan. 10, 2012, in the Korean Intellectual Property Office, the disclosure of which is incorporated herein in its entirety by reference.

### BACKGROUND

1. Field

The present disclosure relates to biomarkers for detecting cancer stem cells or circulating cancer cells that have undergone epithelial-mesenchymal transition, methods of isolating cancer stem cells or circulating cancer cells that have undergone epithelial-mesenchymal transition, and related methods, compositions, and kits.

2. Description of the Related Art

Cancer metastasis, the release and migration of single tumor cells via blood vessels is a major cause of cancer-related morbidity and mortality. Circulating tumor cells (CTCs), cancer cells present in the blood stream of a subject, are believed to play an important role in cancer metastasis. Additionally, some CTCs are likely cancer stem cells, one of the most important subjects of recent cancer research. However, CTCs are delicate, rare, and cannot be easily obtained with traditional biopsies, making their detection and quantification difficult.

As a result, anti-cancer drugs are administered to most patients without confirming the presence or absence of CTCs, which can lead to over-treatment. Selective administration of anti-cancer drugs according to the presence or absence of CTCs, or according to the molecular properties of CTCs, would allow for customized administration of drugs and improve the efficacy of cancer therapy.

Thus, there is a strong need for improved methods of detecting cancer cells, particularly CTCs, and methods of detecting cancer or metastasis in a subject.

### SUMMARY

The invention provides a method of detecting a cancer cell, particularly a cancer stem cell or circulating tumor cell that has undergone epithelial-mesenchymal transition, comprising determining the level of caveolin-1 expressed by a sample cell, and comparing the level of caveolin-1 expressed by the sample cell to a control, wherein higher expression of caveolin-1 by the sample cell indicates that the sample cell is a cancer cell.

The invention further provides a method of detecting cancer or metastasis in a subject, which method comprises detecting a cancer stem cell or circulating tumor cell in a sample as provided herein, wherein the sample is a biological sample from the subject, and the detection of a cancer stem cell or circulating tumor cell that has undergone epithelial-mesenchymal transition is indicative of cancer or metastasis in the subject.

Also provided is a method of isolating a cancer stem cell or a circulating cancer cell that has undergone epithelial-mesenchymal transition from a biological sample, the method comprising contacting the biological sample with an antibody or antibody fragment that specifically binds caveolin-1, wherein the antibody or antibody fragment binds to caveolin-1 on the surface of the cancer stem cell or circulating tumor cell that has undergone epithelial-mesenchymal transition to provide an antibody-bound or antibody-fragment-bound cancer stem cell or circulating tumor cell; and removing the antibody-bound or antibody-fragment-bound cancer stem cell or circulating tumor cell from the sample.

### BRIEF DESCRIPTION OF THE DRAWINGS

FIG. 1 is a western blot showing expression of Caveolin-1 in 9 cancer cell lines.

FIGS. 2A, 2B, and 2C are images showing immunocytochemistry and western blot results confirming that epithelial-mesenchymal transition was induced in MCF7 cell lines. “Sphere” denotes epithelial-mesenchymal transition-induced cells.

FIG. 3 is an image comparing the binding affinity of beads with a Caveolin-1 antibody bound thereto to a MCF-7 cell that has undergone epithelial-mesenchymal transition with the binding affinity of the beads to a normal cancer cell that has not undergone epithelial-mesenchymal transition.

FIG. 4 is a western blot showing expression levels of Caveolin-1, Snail, ALDH1, CD133, and CD44 in epithelial-mesenchymal transition-induced MCF7 cells.

### DETAILED DESCRIPTION

Provided herein is a biomarker for detecting a cancer stem cell or a circulating cancer cell that has undergone epithelial-mesenchymal transition, the biomarker including Caveolin-1 having a relatively higher expression level in a cancer cell than a normal cell.

Epithelial-mesenchymal transition (EMT) is a phenomenon occurring in normal embryonic development, and a process whereby cells lose their epithelial phenotype and undergo transition to a mesenchymal phenotype with high levels of cell motility. If cells undergo irreversible EMT, however, this causes heart failure, liver failure, renal failure, and vascular dysfunction, and is also known to be related to tumor metastasis. Epithelial cells have an adhesion protein on a contact surface between epithelial cells or between an epithelial cell and a matrix, maintain different polarities at upper and basal portions of epithelial cells due to a cytoskeletal structure, and act as a barrier or have secretion and absorption functions. On the contrary, mesenchymal cells each independently migrate, have no polarity, and form connective tissues or extracellular matrixes. When epithelial cells undergo EMT, they lose their polarity, shapes thereof are changed from square to fibroblast type, the number of epithelial cell markers decreases, and the number of mesenchymal cell markers increases. Recent studies have found that EMT plays various roles in regeneration and fibrosis of tissues and tumor development and metastasis other than embryonic histogenesis and differentiation.

Caveolin-1 is a protein encoded by CAV1 that is present in caveolae, invaginations of the cell membrane found in most types of human cells, and is involved in promoting cell cycle progression. Cancer cells that have undergone epithelial-mesenchymal transition, such as CTC or cancer stem cells, show increased expression of caveolin-1. Accordingly, caveolin-1 may be used as a biomarker for the detection of a cancer cell, particularly a CTC or cancer stem cell, or for the detection of cancer or metastasis in a subject.

In one embodiment, the invention provides a method of detecting a cancer cell, particularly a CTC or cancer stem cell, in a sample comprising determining the level of caveolin-1 expressed by a sample cell, and comparing the level of caveolin-1 expressed by the sample cell to a control, wherein higher expression of caveolin-1 by the sample cell indicates that the sample cell is a cancer cell. The invention further provides a method of detecting cancer or metastasis in a subject, comprising obtaining a biological sample from a subject, and analyzing the biological sample to detect a cancer cell, wherein the detection of a cancer cell is indicative of cancer or metastasis in the subject.

The cancer cell may be a cancer cell such as a CTC, a cancer stem cell, and/or a cancer cell that has undergone epithelial-mesenchymal transition.

The caveolin-1 may be the complete caveolin-1 protein (SEQ ID NO: 1) or a fragment thereof that can be detected by an antibody or antibody fragment (e.g., an immunogenic fragment of caveolin-1. The caveolin-1 may be expressed on the surface of the cell.

The level of caveolin-1 expressed by the sample cell may be determined using any method known in the art. For instance, the level of caveolin-1 expressed by the sample cell may be determined using an antibody or antigen-binding fragment thereof that specifically binds caveolin-1 or a fragment thereof.

According to another embodiment of the present invention, there is provided a kit for detecting a cancer stem cell or a circulating cancer cell that has undergone epithelial-mesenchymal transition in a sample, the kit including an antibody specifically binding to Caveolin-1 or a fragment thereof or an antigen binding fragment thereof and a support, such as a magnetic or non-magnetic bead or other surface to which a sample may be applied. The antibody may, optionally, be bound to the support.

The antibody may be a polyclonal antibody. The polyclonal antibody may be produced by injecting a biomarker protein or a fragment thereof, as an immunogen, to a foreign host, according to any method known in the art. The foreign host may include mammals such as mice, rats, sheep, and rabbits. The immunogen may be injected through intramuscular, intraperitoneal or subcutaneous injection, and may be administered together with an adjuvant to improve antigenicity. Afterwards, blood is periodically collected from the foreign host to obtain blood serum showing improved titer and antigenic specificity, from which an antibody is separated and purified.

Alternatively, a monoclonal antibody can be used. The monoclonal antibody may be produced by a technology for producing immortalized cell lines by any method known in the art. The monoclonal antibody may be produced by a technology for producing immortalized cell lines by fusion known in the art. Hereinafter, the production method will be simply described. First, an appropriate amount (approximately 10 μg) of pure protein is obtained, and is immunized into Balb/C mouse. Otherwise, a polypeptide fragment of the pure protein is synthesized, bound to bovine serum albumin, and immunized into a mouse. Then, antigen-producing lymphocyte separated from the mouse is fused with myeloma of a human or a mouse to produce immortalized hybridoma. Then, ELISA method is used to select and proliferate only a hybridoma cell producing a desired monoclonal antibody, and the monoclonal antibody may be separated and purified from the culture.

The monoclonal antibody may be variously used for an immunoassay kit (e.g., ELISA, antibody coated tube test, lateral-flow test, potable biosensor), and may be also used to develop a protein chip having a detection spectrum for various cancer cells through development of an antibody showing higher specificity and sensitivity.

Antibody fragments include, without limitation, an scFv fragment, a (scFv)₂ fragment, a Fab fragment, a Fab′ fragment, and a F(ab′)₂ fragment. The term “antibody fragment” or “antigen binding fragment” used herein refers to fragments of an intact immunoglobulin, and any part of a polypeptide including antigen binding regions. A Fab fragment has one antigen binding site and contains the variable regions of a light chain and a heavy chain, the constant region of the light chain, and the first constant region C_{H1} of the heavy chain. A Fab′ fragment is different from the Fab fragment in that the Fab′ fragment additionally includes the hinge region of the heavy chain, including at least one cysteine residue at the C-terminus of the heavy chain C_{H1} region. A F(ab′)₂ fragment is produced whereby cysteine residues of the Fab′ fragment are joined by a disulfide bond at the hinge region. An Fv fragment is the minimal antibody fragment having only heavy chain variable regions and light chain variable regions, and a recombinant technique for producing the Fv fragment is well known in the art. Two-chain Fv fragments may have a structure in which heavy chain variable regions are linked to light chain variable regions by a non-covalent bond. Single-chain Fv fragments generally may have a dimer structure as in the two-chain Fv fragments in which heavy chain variable regions are covalently bound to light chain variable regions via a peptide linker or heavy and light chain variable regions are directly linked to each other at the C-terminus thereof. The antigen binding fragment may be obtained using a protease (for example, a whole antibody is digested with papain to obtain Fab fragments, or is digested with pepsin to obtain F(ab′)₂ fragments), and may be prepared by a genetic recombinant technique.

The process of determining the level of caveolin-1 expressed by the sample cell may be performed by immunoassay. The immunoassay may be prepared according to immunoassay or immunostaining protocols which have been conventionally developed. Examples of immunoassay or immunostaining methods include radioimmunoassay, radioimmunoprecipitation, immunoprecipitation, enzyme-linked immunosorbent assay (ELISA), capture-ELISA, inhibition or competition assay, sandwich analysis, flow cytometry, immunofluorescence staining, and immunoaffinity purification, but are not limited thereto. For example, in an embodiment of a radioimmunoassay method, a radioisotope-labeled antibody may be used to detect caveolin-1. The radioisotope may be, for example, C¹⁴, I¹²⁵, P³², or S³⁵.

In an embodiment of an ELISA method, the method may include: (i) coating a surface of a solid substrate with a blood sample of each of a normal person and a subject likely to have cancer; (ii) contacting the blood sample with an antibody specifically binding to caveolin-1 or a fragment thereof as a primary antibody to induce an antigen-antibody reaction; (iii) reacting the resultant product obtained by (II) process with a secondary antibody with an enzyme conjugated thereto; and (iv) detecting the activity of the enzyme.

The solid substrate may be a hydrocarbon polymer such as polystyrene or polypropylene, glass, a metal, or a gel. For example, the solid substrate may be a microtiter plate. The enzyme conjugated to a secondary antibody may be an enzyme catalyzing a colorimetric, fluorometric, luminescence or infra-red reactions, but is not limited thereto. For example, the enzyme may be alkaline phosphatase, β-galactosidase, horseradish peroxidase, luciferase, or Cytochrome P₄₅₀. When alkaline phosphatase is used, bromo-chloro-indolyl-phosphate (BCIP), nitro blue tetrazolium (NBT), naphthol-AS-B1-phosphate, or enhanced chemifluorescence (ECF) may be used as a substrate. When horseradish peroxidase is used, chloronaphtol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), hypersensitive reaction solution (HYR: p-phenylenediamine-HCl and pyrocatechol), tetramethylbenzidine (TMB), 2,2′-Azine-di[3-ethylbenzthiazoline sulfonate] (ABTS), o-phenylenediamine (OPD) and naphtol/pyronin, glucose oxidase and t-nitroblue tetrazolium (t-NBT), or m-phenzaine methosulfate (m-PMS) may be used as a substrate.

In an embodiment of a capture-ELISA method, the method may include: (i) coating a surface of a solid substrate with an antibody specifically binding to daveolin-1 or a fragment thereof as a capturing antibody; (ii) contacting the capturing antibody and a blood sample of a subject likely to have cancer to induce an antigen-antibody reaction; (iii) reacting the resultant product obtained by (II) process with a detecting antibody with a signal-generating label attached thereto and specifically binding to caveolin-1; and (iv) detecting the signal generated from the label. The detecting antibody may have a label generating a detectable signal. The label may be a chemical label such as biotin; an enzymatic label such as alkaline phosphatase, β-galactosidase, horseradish peroxidase and Cytochrome P₄₅₀; a radioactive label such as C¹⁴, I¹²⁵, P³² and S³⁵; a fluorescent label such as fluorescein; a luminescent label; chemiluminescent label; or a fluorescence resonance energy transfer (FRET) label, but is not limited thereto.

The final measurement of enzyme activities or signals in the ELISA method and the capture-ELISA method may be performed by any method known to one skilled in the art to enable quantitative or qualitative analysis of caveolin-1. For example, signals may be detected easily by streptavidin in the case of a biotin-labeled antibody and by luciferin in the case of a luciferase-labeled antibody.

A microchip or an automated microarray system may be used to detect an antigen for the antibody by immobilizing an antibody specifically binding to caveolin-1 or a fragment thereof on a microchip and then reacting the antibody with a biological sample isolated from a subject. By using a microchip or an automated microarray system, a large amount of a biological sample can be analyzed at once.

The control may be a cancer cell that has not undergone epithelial-mesenchymal transition, or a cell that is not a cancer cell (e.g., a “normal” cell). The control may be a biological sample (e.g., blood or other body fluid, tissue, cell, etc.) from a subject that does not have cancer. The control also may be a pre-established value representing the expression of caveolin-1 in a normal, non-cancerous sample or in a population of such samples. A positive control can be used instead of, or in addition to, a negative control, wherein the positive control may be a cancer cell that known to have undergone epithelial-mesenchymal transition, or a known cancer cell, circulating tumor cell, or cancer stem cell that exhibits increased caveolin-1 expression. The positive control may be a biological sample (e.g., blood or other body fluid, tissue, cell, etc.) from a subject known to have cancer, particularly metastatic cancer. The control also may be a pre-established value representing the expression level of caveolin-1 in a cancerous sample, particularly a sample with CTCs or cancer stem cells, or in a population of such samples

The method of detecting a cancer cell, particularly a CTC or cancer stem cell, in a sample can be used for any purpose, including the detection or diagnosis of cancer or cancer metastasis, or tracking the progression of cancer or cancer metastasis. In particular, the detection of a cancer cell, particularly a CTC or cancer stem cell, in a sample is indicative of cancer or cancer metastasis in the subject from which the sample was obtained. Cancer may be diagnosed, for example, by analyzing the intensity of a final signal from an immunoassay. For instance, when the signal for caveolin-1 from a sample (e.g., blood) of a subject likely to have cancer is stronger (increased expression) than the corresponding signal from a normal control sample, the subject may be diagnosed as having cancer, cancer metastasis, or a likelihood of developing metastasis.

After detecting a circulating tumor cell or cancer stem cell, the method may further include isolating the cell from the biological sample. For example, the isolating process may be performed by centrifugation, filtration, or chromatography, optionally using fluorescently labeled antibodies or antibody fragments specific to caveolin-1. Alternatively, or in addition, the CTC or cancer stem cell that has undergone epithelial-mesenchymal transition and expresses caveolin-1 on the surface thereof may be captured and/or isolated from a sample using an antibody or antibody fragment specific to caveolin-1. For instance, a sample containing such cells can be combined or contacted with an antibody or antibody fragment specific to caveolin-1 such that the antibody or antibody fragment binds to the cells. The cell/antibody complex can then be isolated from the sample using any technique. For instance, the antibodies or antibody fragments can be immobilized on a support (beads, column, substrate, etc.), and the sample passed over the immobilized antibodies. Or, the antibodies or antibody fragments can be conjugated to an affinity tag (labeled beads, magnetic beads, fluorescent or other detectable tags), and isolated on that basis. Such a method can be performed in conjunction with a method of detecting the CTC or cancer stem cell as described herein, or separate and apart from any method of detection.

The isolated cancer cells may be cultured using a culturing method well known to one of ordinary skill in the art, thereby being suitable for use in experiments.

According to one embodiment, the sample (biological sample) may be any sample containing a cancer cell. For example, the biological sample may be selected from the group consisting of blood, bone marrow fluid, lymph fluid, saliva, lachrymal fluid, urine, mucous fluid, amniotic fluid, and combinations thereof, but is not limited thereto.

If cancer or metastasis is detected in the subject, the method may further comprise treating the cancer or metastasis with an appropriate cancer therapy.

The invention additionally provides a kit for the detection of a cancer cell. The kit may be manufactured according to any method known in the art, and may typically include freeze-dried antibody and buffer, a stabilizer, inactive protein, and the like.

One or more embodiments of the present invention will now be described more fully with reference to the following examples. However, these examples are provided only for illustrative purposes and are not intended to limit the scope of the present invention.

One or more embodiments of the present invention will now be described more fully with reference to the following examples. However, these examples are provided only for illustrative purposes and are not intended to limit the scope of the present invention.

### Example 1

### Detection of Protein Expressed in Breast Cancer Cell Lines Having a Low Expression Level of EpCAM Through Microarray

51 types of breast cancer cell lines were divided according to whether or not EpCAM was expressed, with reference to Cancer Cell, 10(6): 515-527 (2006), and proteins expressed on surfaces of the breast cancer cell lines were analyzed using microarray data. From the microarray data, it was confirmed that 20 breast cancer cell lines including R 75-1, SKBR3, MCF-7, and the like have a high expression level of EpCAM, and it was confirmed that 31 breast cancer cell lines including MDA231, MDA436, MCF10A, and the like have a low expression level of EpCAM. As a result of analysis, it was confirmed that the breast cancer cell lines with a low expression level of EpCAM had a high expression level of caveolin-1.

### Example 2

### Confirmation of Expression Level of Caveolin-1 According to Cancer Cell Lines

An expression level of caveolin-1 in various kinds of cancer cell lines was confirmed by western blotting. 9 types of cancer cell lines (purchased from ATCC (American Type Culture Collection)) including breast cancer cell lines (i.e., ZR75-1, SKBR3, MCF7, MDA-MB-231, MDA-MB-436, MCF10A) and prostate cancer cell lines (i.e., PC3, LnCAP, DU145) were cultured in a DMEM medium in a 100 mm culture dish, and then cell extracts were obtained therefrom. 20 μg of each cell extract was isolated using a Novex NuPAGE Bis-Tris Electrophoresis System (Invitrogen) and then transferred on a nitrocellulose membrane (Invitrogen, cat.no #LC2006). Each membrane was blocked in 3% skim milk for 1 hour and then reacted with Caveolin-1 antibodies (AbCAM, cat.no #2910) diluted to 1:1000 at 4° C. for 18 hours or longer. Then, the resultant membrane was fully washed with a TBS-T solution to remove unreacted antibodies, and each resultant membrane was reacted with goat anti-rabbit IgG-horseradish peroxidase (HRP) at room temperature for 1 hour. Afterwards, the resulting membranes were fully washed with a TBS-T solution, and a peroxidase substrate solution (Thermo Scientific Pierce ECL Western Blotting Substrate, cat.no #32106) was added thereto to generate fluorescence. The generated fluorescence was measured to compare the expression levels of caveolin-1 in the 9 types of breast cancer cell lines with one another.

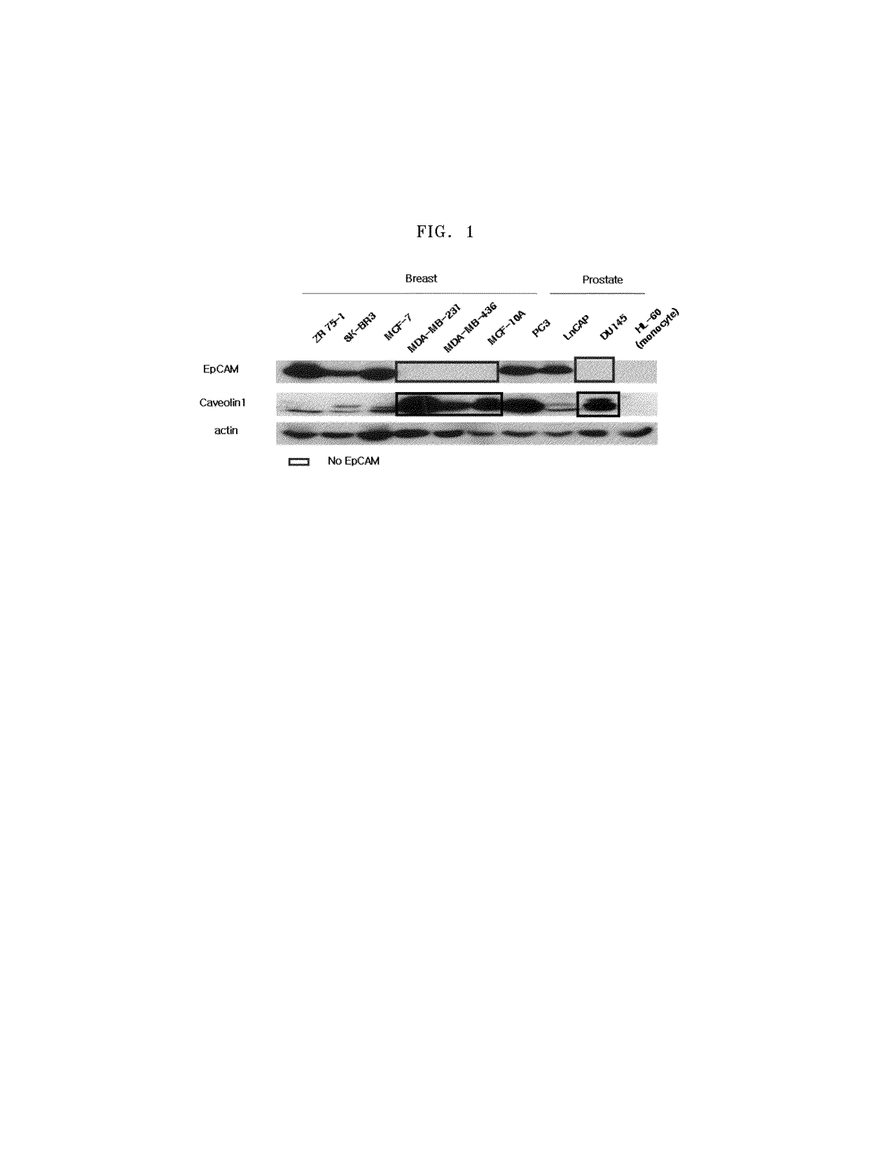

As a result, as shown in FIG. 1, it was confirmed that the MDA-MB-231, MDA-MB-436 and DU145 cell lines that had no expression of EpCAM from among the 9 types of breast cancer cell lines had a strong expression level of caveolin-1.

### Example 3

### Production of Beads with Caveolin-1 Antibodies Bound Thereto

COOH polystyrene beads having a diameter of 1 or 3 μm were treated with EDC(N-hydroxysuccinimide)/NHS(1-ethyl-3-[3-dimethylaminopropyl]carbodiimide hydrochloride), the treated beads were added to the prepared PBS solution, 0.65 mg/ml of antibodies specifically binding to caveolin-1 were added thereto, and the resulting solution was slowly shaken at room temperature for 2 hours, thereby completing the production of beads to which antibodies specifically binding to caveolin-1 were bound.

### Example 4

### Artificially Induced Epithelial-Mesenchymal Transition in Breast Cancer Cell Lines

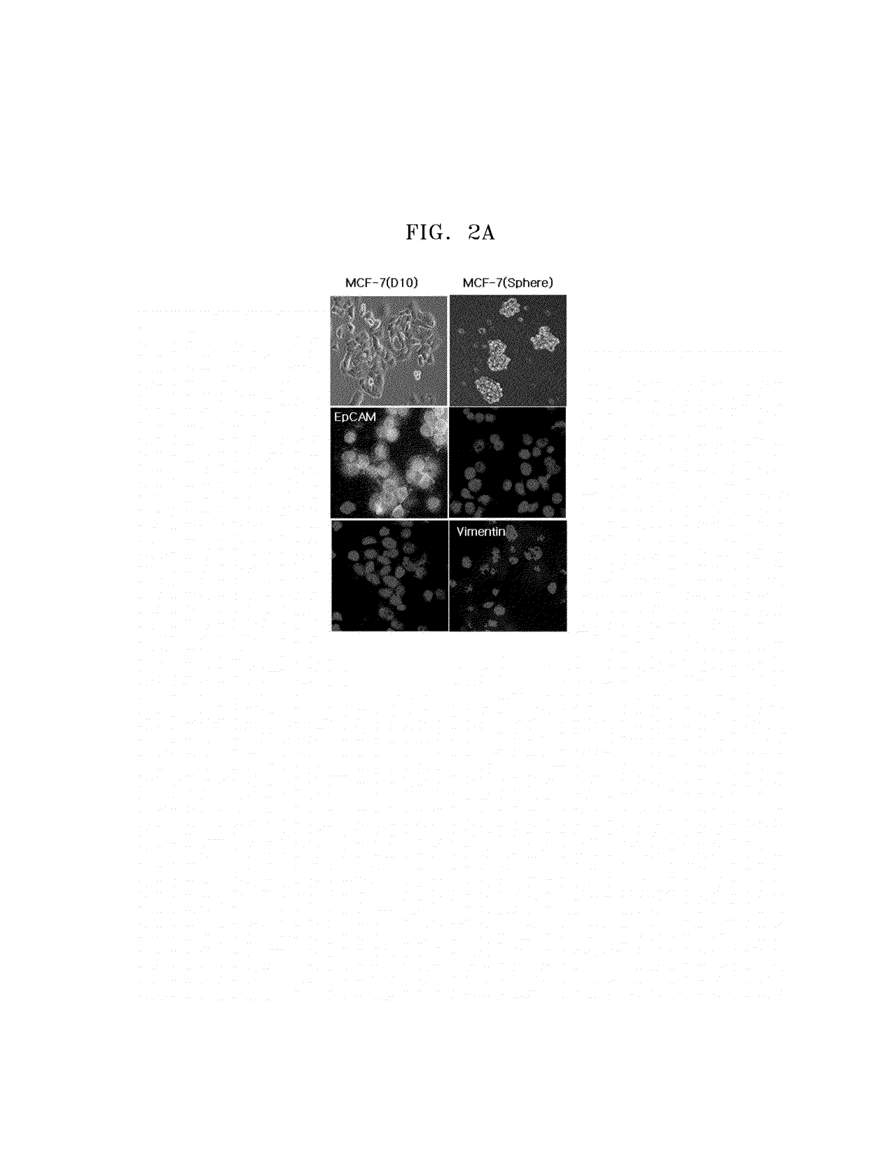

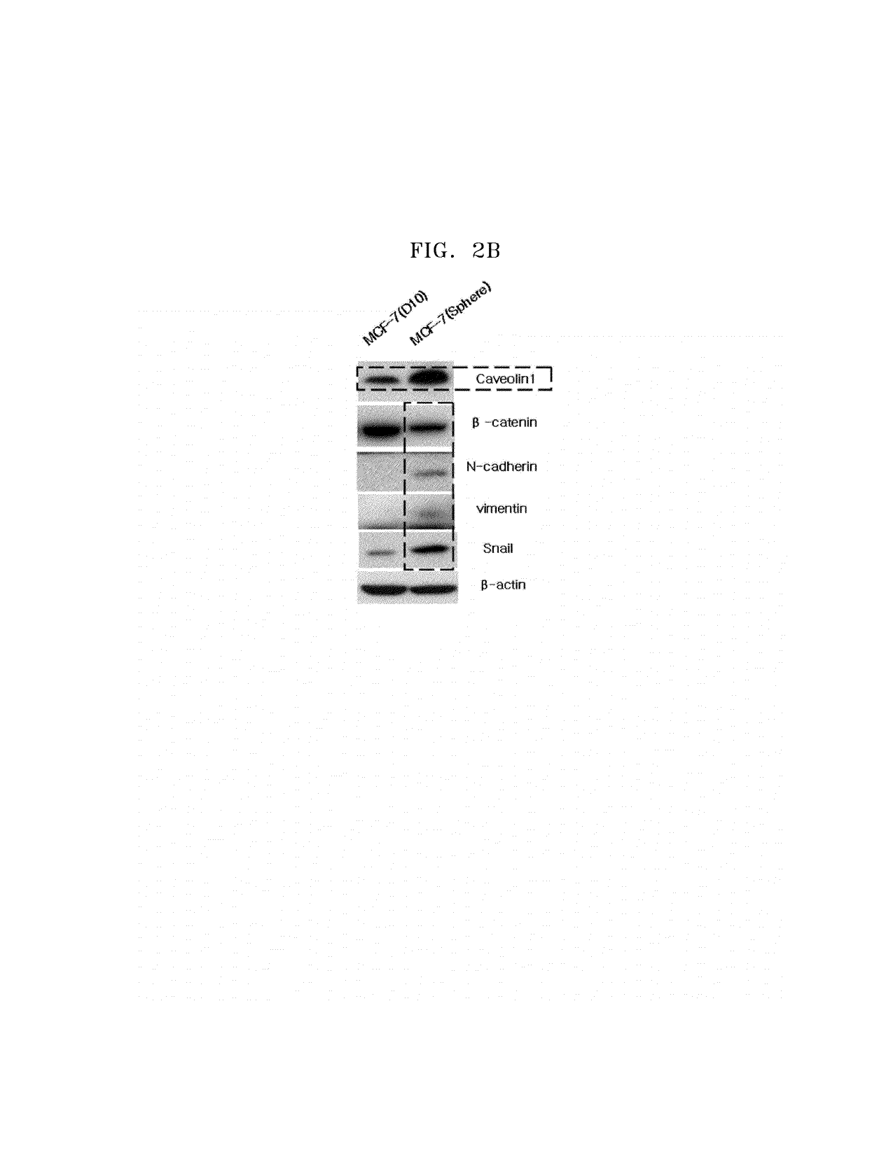

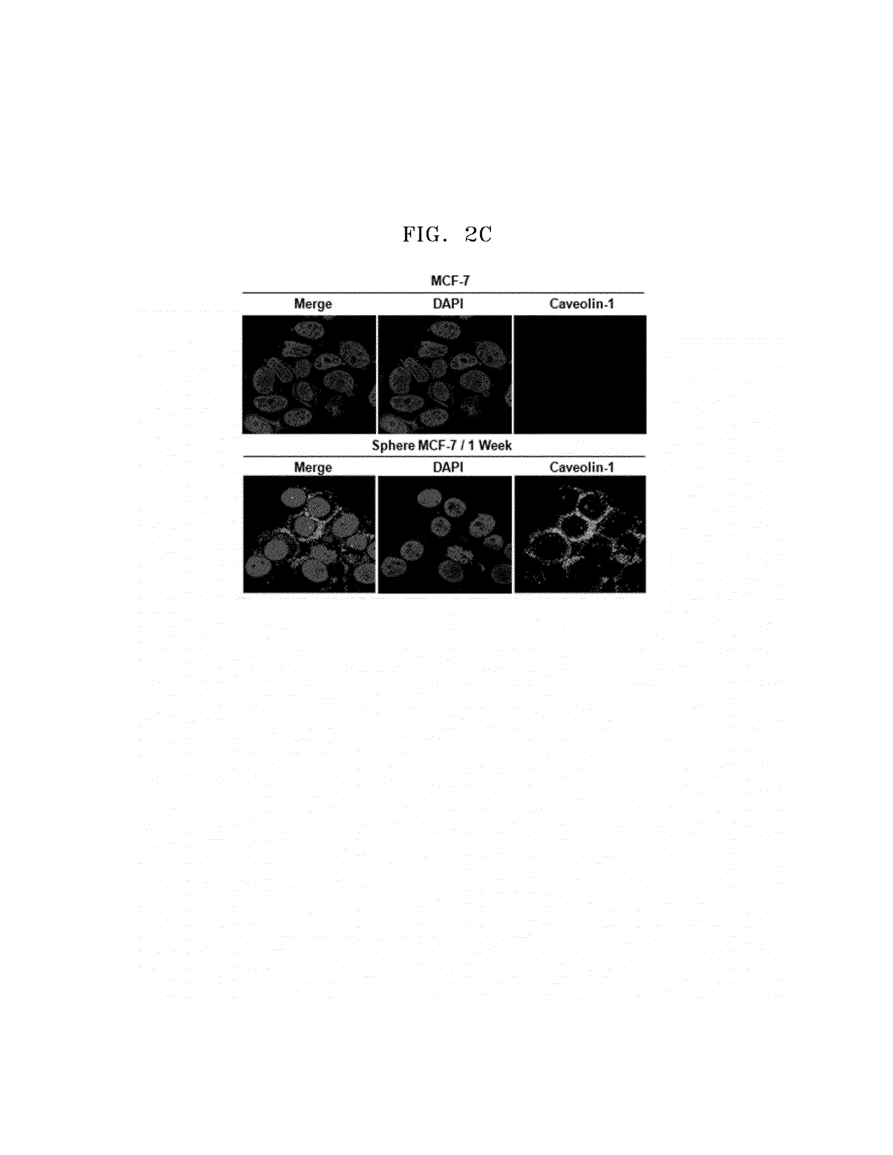

To induce epithelial-mesenchymal transition in breast cancer cell lines MCF7, a mommosphere culture method described below was used instead of the existing attachment culture (DMEM+10% FBS) method. A medium containing DMEM-F12, 1×B27, 20 ng/ml FGF, 20 ng/ml EGF, and 5 ug/ml insulin was used as a culture medium, and bacteria cells (2×10⁵ cells/ml) were inoculated in a 100 mm dish and then cultured for 1 week. After the culturing process, immunocytochemistry was performed on the cultured bacteria cells. As a result, as shown in FIG. 2A, it was confirmed that there was no expression of EpCAM, which is an epithelial marker, and the expression of vimentin, which is a mesenchymal marker, increased. In addition, as a result of western blotting, as shown in FIG. 2A, it was confirmed that epithelial-mesenchymal transition was induced by confirming that the amount of β-catein, which is a protein known to have decreased expression when epithelial-mesenchymal transition was induced, decreased, and the amounts of snail, N-cadherin, and vimentin, which are proteins known to have increased expression when epithelial-mesenchymal transition was induced, increased. In this regard, as seen in FIGS. 2B and 2C, it was confirmed that the expression level of caveolin-1 significantly increased when epithelial-mesenchymal transition was induced.

### Example 5

### Confirm the Binding Affinity of Beads with a Caveolin-1 Antibody Bound Thereto to Circulating Cancer Cells that Had Undergone Epithelial-Mesenchymal Transition

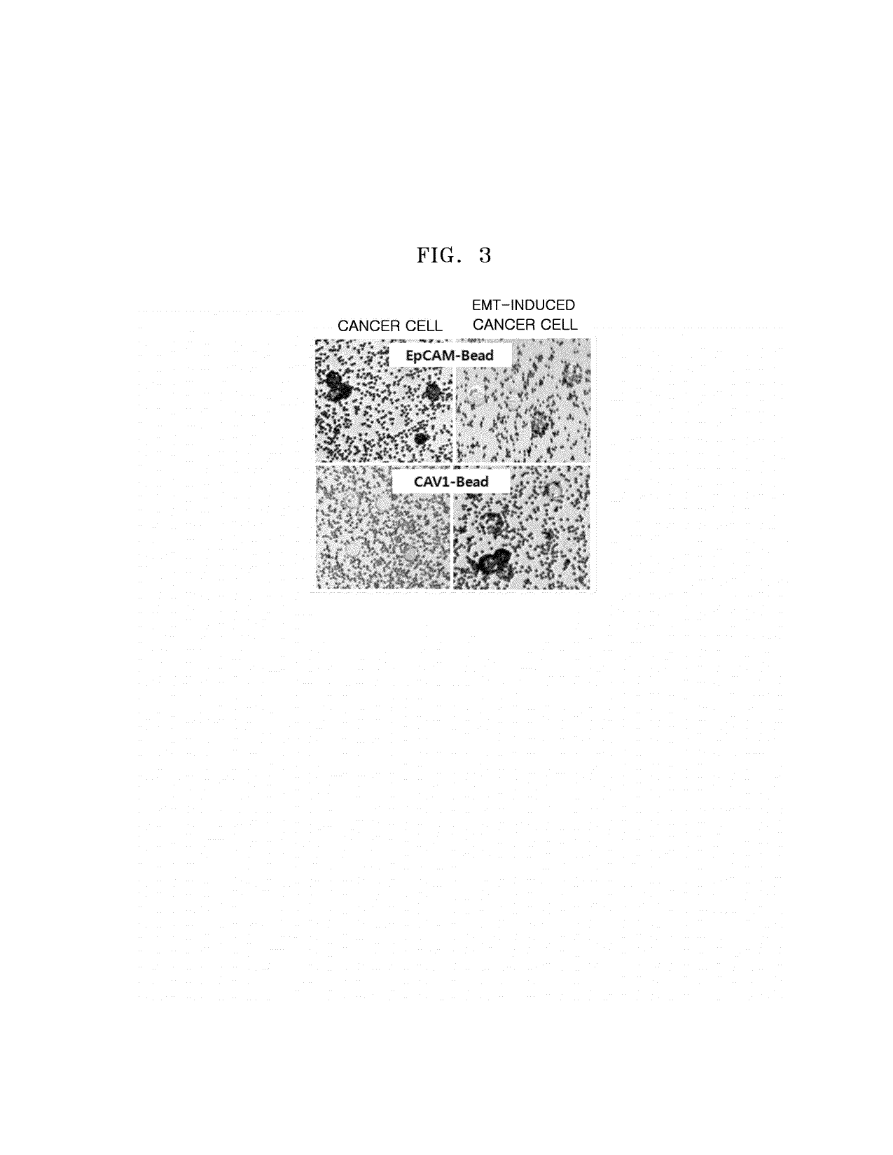

First, to confirm whether or not caveolin-1 was expressed on surfaces of MCF-7 cell lines that had undergone epithelial-mesenchymal transition, as in Example 4, epithelial-mesenchymal transition was induced in breast cancer cell lines MCF7 with a high expression level of EpCAM. Subsequently, 30 μl of the beads with a caveolin-1 antibody bound thereto which were prepared according to Example 3 was added to 1×10⁵ MCF-7 cell lines suspended in a DMEM medium and left for 1 hour. Then, whether the beads were bound to the MCF-7 cell lines was confirmed through the fluorescence intensity of fluorescein by using a fluorescence microscope (Olympus IX-81). In this example, beads with an EpCAM antibody bound thereto were used as a control. As a result, as seen in FIG. 3, it was confirmed that the binding of the beads with a caveolin-1 antibody bound thereto to the MCF-7 cell lines that had undergone epithelial-mesenchymal transition notably increased as compared to normal cancer cells. Then, the beads with caveolin-1 antibodies bound thereto were separated by centrifugation.

### Example 6

### Confirmation of Caveolin-1 as a Marker for Circulating Cancer Cells that have Undergone Epithelial-Mesenchymal Transition or Cancer Stem Cells

To confirm that caveolin-1 can be used as a marker for circulating cancer cells that have undergone epithelial-mesenchymal transition or cancer stem cells, western blotting was used to confirm whether or not snail known as a marker for circulating cancer cells that have undergone epithelial-mesenchymal transition and ALDH1, CD133, and CD44, which are known as markers for cancer stem cells, was expressed in breast cancer cell lines MCF7 in which epithelial-mesenchymal transition was induced. The western blotting process may be performed in the same manner as in Example 2, except that a caveolin-1 antibody (AbCAM, cat.no #2910), a Snail antibody (Cell signaling cat.no #3879), an ALDH1 antibody (AbCAM, cat.no #23375), a CD133 antibody (AbCAM, cat.no #27699), and a CD44 antibody (Cell signaling cat.no #5640) were respectively used for the markers as a primary antibody.

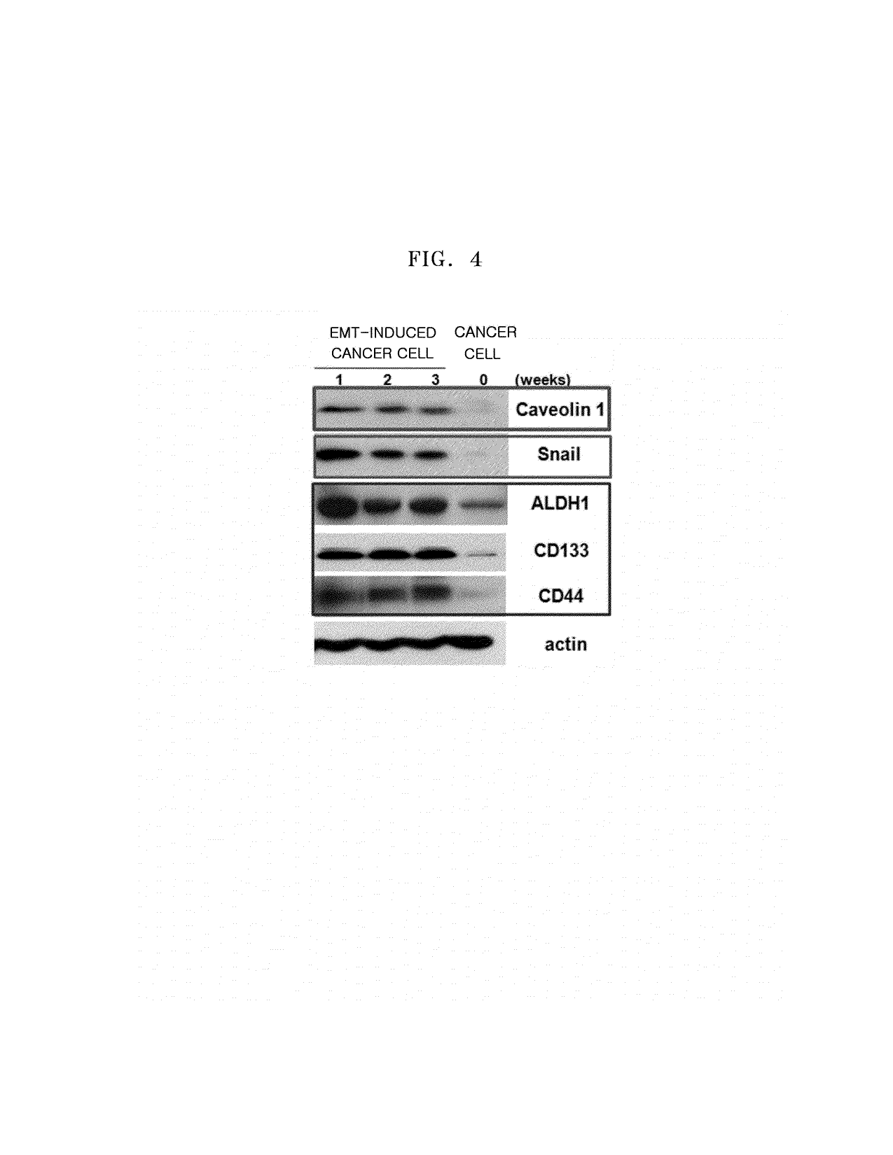

As a result, as seen in FIG. 4, it was confirmed that the expression levels of caveolin-1, snail, ALDH1, CD133, and CD44 increased in the breast cancer cell lines MCF7 in which epithelial-mesenchymal transition was induced. In view of the results that caveolin-1 exhibited the same pattern of increase in expression as that of snail, ALDH1, CD133, and CD44, this indicates that caveolin-1 may also be used as a marker for circulating cancer cells that have undergone epithelial-mesenchymal transition or a marker for cancer stem cells.

What is claimed is:

1. A method of detecting a cancer stem cell or circulating tumor cell that has undergone epithelial-mesenchymal transition in a sample, the method comprising:
determining the level of caveolin-1 expressed by a cell of the sample, and
comparing the level of caveolin-1 expressed by the cell of the sample to a control,
wherein higher expression of caveolin-1 by the cell of the sample indicates that the cell of the sample is a cancer stem cell or circulating cancer cell that has undergone epithelial-mesenchymal transition.

2. The method of claim 1, wherein the level of caveolin-1 expressed by the sample cell is determined using an antibody or antigen-binding fragment thereof that specifically binds caveolin-1 or a fragment thereof.

3. The method of claim 2, wherein the antibody is a monoclonal antibody or a polyclonal antibody.

4. The method of claim 2, wherein the antigen-binding fragment is selected from the group consisting of an scFv fragment, a (scFv)₂ fragment, a Fab fragment, a Fab′ fragment, and a F(ab′)₂ fragment.

5. The method of claim 1, wherein the control is a cancer cell that has not undergone epithelial-mesenchymal transition, or a cell that is not a cancer cell.

6. The method of claim 1, further comprising isolating the cancer cell.

7. A method of detecting cancer or cancer metastasis in a subject comprising detecting a cancer stem cell or circulating tumor cell in a sample according to claim 1,
wherein the sample is a biological sample from the subject, and the detection of a cancer stem cell or circulating tumor cell that has undergone epithelial-mesenchymal transition is indicative of cancer or metastasis in the subject.

8. The method of claim 7, wherein the biological sample is selected from the group consisting of blood, bone marrow fluid, lymph fluid, saliva, lachrymal fluid, urine, mucous fluid, amniotic fluid, and combinations thereof.

9. The method of claim 7, further comprising treating the cancer or metastasis.

10. A kit for detecting a cancer stem cell or a circulating cancer cell that has undergone epithelial-mesenchymal transition, the kit comprising an antibody specifically binding to Caveolin-1 or an antigen binding fragment thereof and a support.

11. A method of isolating a cancer stem cell or a circulating cancer cell that has undergone epithelial-mesenchymal transition from a biological sample, the method comprising
contacting the biological sample with an antibody or antibody fragment that specifically binds caveolin-1, wherein the antibody or antibody fragment binds to caveolin-1 on the surface of the cancer stem cell or circulating tumor cell that has undergone epithelial-mesenchymal transition to provide an antibody-bound or antibody-fragment-bound cancer stem cell or circulating tumor cell;
and removing the antibody-bound or antibody-fragment-bound cancer stem cell or circulating tumor cell from the sample.

12. The method of claim 11, wherein the antibody is immobilized on a support or conjugated to an affinity tag.

13. The method of claim 11, wherein the biological sample is selected from the group consisting of blood, bone marrow fluid, lymph fluid, saliva, lachrymal fluid, urine, mucous fluid, amniotic fluid, and combinations thereof.
